(19) United States
(12) Patent Application Publication
(10) Pub. No.: US 2008/0300575 A1
(43) Pub. Date: Dec. 4, 2008

(54) Anal ointment applicator

(75) Inventors: Iain G.M. Cleator, Vancouver (CA); Marc Gaetan Morin, West Vancouver (CA)

Correspondence Address: NIXON & VANDERHYE, PC, 901 NORTH GLEBE ROAD, 11TH FLOOR, ARLINGTON, VA, 22203, US

(73) Assignee: 0696578 B.C. LTD. Incorporation #BC0696578, West Vancouver (CA)

(21) Appl. No.: 12/213,058

(22) Filed: Jun. 13, 2008

Related U.S. Application Data
(62) Division of application No. 11/498,860, filed on Aug. 4, 2006.

(51) Int. Cl. A61M 31/00

(52) U.S. Cl. 604/514; 604/57

(57) ABSTRACT

Medicinal applicator suitable for delivering medicinal material in a measured dose and concentration to a body cavity, having an elongate support body with an external surface and a blunt distal end to facilitate insertion into a body cavity and a support element receiving zone located towards the distal end for receiving the support element. The support element receiving zone is configured to receive a correspondingly shaped support element for carrying medicinal material, whereby upon insertion of the elongate body into the body cavity, medicinal material supported on the support element located in the receiving zone is released to a desired location within the cavity.

The present invention relates to a device for insertion into and application of medication to the anal canal.

### BACKGROUND OF THE INVENTION

Anal fissure is a painful tear in the lining of the anal canal, which causes many problems for the patient. For example, bowel movement is painful, as is walking. Painful spasms (anal spasm) of the surrounding muscle are common, and bleeding, sometimes massive, typically occurs. Anal fissure also provides possible access to bacteria from stool, causing sepsis locally and elsewhere.

A surgery is often employed to partially divide the muscle surrounding the anal canal (the anal sphincter) and relax the smooth muscle enough to permit healing. Unfortunately, this surgery sometimes results in loss of control of bowel movements.

Use of an ointment, for example a nitroglycerin ointment or diltiazem ointment (an ACE inhibitor), locally to relax the smooth muscle results in healing in over 80% of patients without the complications, risks and expense of surgery. Currently, this ointment is applied with the finger to the anal area. If the correct amount reaches the fissure, the sphincter is relaxed, relieving the pain of the anal spasms that keep the patient awake. Healing typically occurs in about 6 weeks. In a fissure which has been present for years, the smooth muscle of the nearby vessels also relaxes, permitting an increased blood supply and healing of the scarred fissure. The anal canal is held closed normally so application of an ointment with the finger results in much of the ointment being swept off on the outside of the anus and, if the usual applicator is used, the ointment goes above the canal into the rectal ampulla and no ointment is deposited on the fissure.

It is difficult to apply the correct amount of the ointment directly to the fissure with the finger. Moreover, the ointment is usually absorbed into the body through the unhealed fissure in the first few days, and can proceed to the heart and circulation (systemic absorption). The application of too much ointment can result in fast pulse, palpitations, faintness and a severe persistent headache. Currently, patients put up with this to get rid of their fissure.

A need exists for a device that can apply the fissure healing ointment directly and evenly on the fissure and which can measure the amount of ointment dispensed. The present invention seeks to satisfy this need.

### SUMMARY OF THE INVENTION

In accordance with one aspect of the present invention, there is provided an applicator for delivering medicinal material suitably in a measured dose and concentration to a body cavity, comprising an elongate support body having an external surface and a blunt distal end to facilitate insertion into a body cavity and a support element receiving zone located towards the distal end for receiving the support element. The support element receiving zone is configured to receive a correspondingly shaped support element for carrying medicinal material, whereby upon insertion of the elongate body into the body cavity, medicinal material supported on a support element located in the receiving zone is released to a desired location within the cavity.

In a further aspect, there is provided a glove applicator for applying material into a body cavity. The glove applicator comprises individual finger receiving elements for receiving respective fingers and thumb of a hand, the forefinger receiving element of which includes a hollow near the distal end thereof for receiving material to be applied within the cavity. Suitably, the hollow has a volume such that a measured dose and concentration may be inserted into the hollow.

In another aspect, there is provided a method of applying a medication to a site of application inside a body cavity using an applicator of the invention, by providing a suitable medication on the support element, inserting the support element in the support element receiving zone, orienting the elongate body to position the aperture in regard to the site of application, and inserting the applicator in the body cavity to apply the medication to the site of application.

### BRIEF DESCRIPTION OF THE DRAWINGS

FIGS. 1a, 1b, 1c, 1d, 1e, 1f, 1g and 1h are views of a first embodiment of an applicator of the invention;

FIG. 2 is a side view from the other side of the embodiment of FIG. 1;

FIGS. 3a, 3b and 3c are views of a further embodiment of an applicator of the invention with slots in each quadrant of the external surface of the elongate support body;

FIGS. 4a and 4b are views of a further embodiment with a plunger and a series of apertures in the upper external surface of the elongate body;

FIGS. 5a, 5b, 5c, and 5d are views of a yet further embodiment of the invention;

FIGS. 6a, 6b, 6c and 6d are views of a further embodiment of the invention;

FIGS. 7a, 7b and 7c are views of a further embodiment of the invention;

FIGS. 8a, 8b and 8c are views of a further embodiment of the invention;

FIGS. 9a, 9b and 9c are views of a further embodiment of the invention;

FIGS. 10a, 10b, 10c, 10d, 10e and 10f are views of a further embodiment of the invention;

FIGS. 11a, 11b, 11c and 11d are views of a further embodiment of the invention;

FIGS. 12a and 12b are side views of a suppository design;

FIGS. 13a and 13b show a glove applicator with a hollow in the forefinger for receiving a measured amount of ointment to be applied to the fissure.

### DETAILED DESCRIPTION OF THE INVENTION

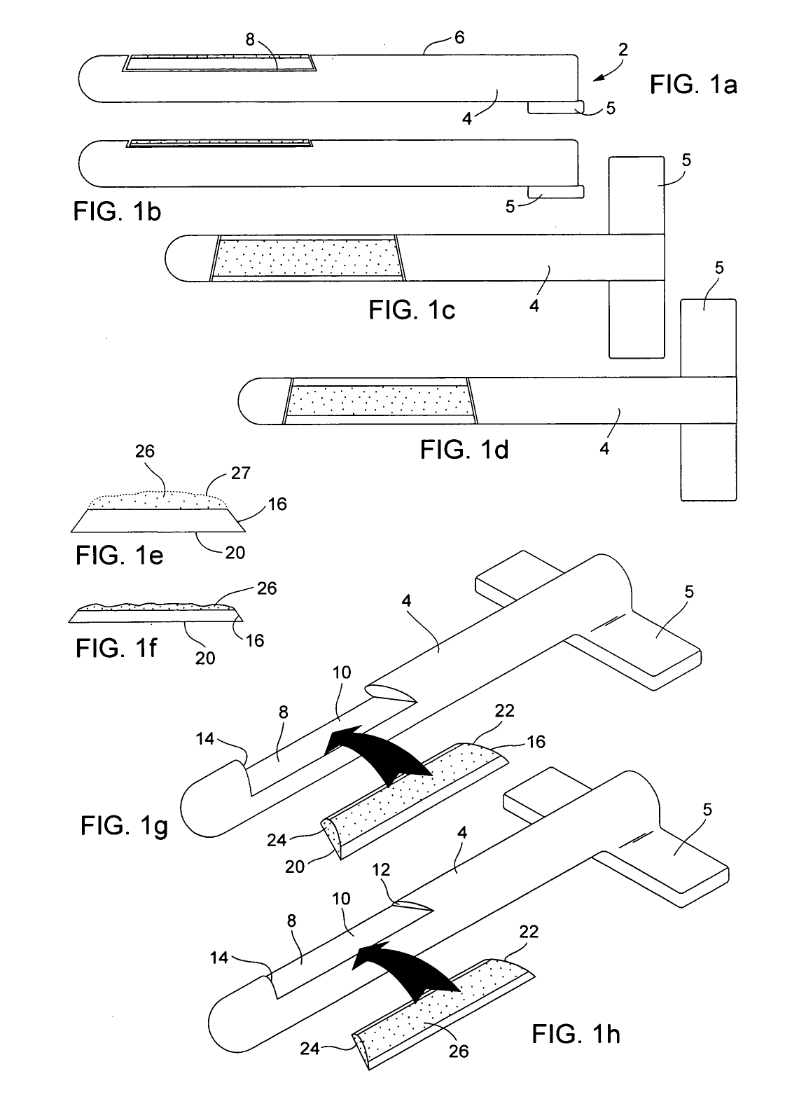

Referring to the drawings, FIG. 1 is a series of views of a first embodiment of an applicator of the invention. FIG. 1a is a side view of the applicator 2 from one side thereof, showing elongate body 4 with an orientation indicator 5 and an external surface 6 containing a support element receiving zone 8 in the form of a slot in an upper portion of the external surface 6. The orientation indicator 5 typically in the form of a handle, and slot 8 are positioned on the elongate body 4 such that, in use, when the handle 5 extends horizontally across the bottom of the elongate body, the slot 8 is disposed on the top of the elongate body. This allows the user to know the orientation of the slot when not visible inside a body cavity. The slot 8 has a base region 10 extending longitudinally of the elongate body 4 with opposed inwardly sloping sidewalls 12, 14 at each end of the base region 10. As seen in FIGS. 1c and 1d, when viewed in plan, the inwardly sloping side walls 12, 14 are arranged obliquely with respect to each other to define a trapezoidal shape when viewed in plan.

The slot 8 is configured to receive a correspondingly shaped support element for carrying medicinal material, whereby upon insertion of the elongate body into the body cavity, medicinal material supported on a support element located in the receiving zone is released to a desired location within the cavity. In one embodiment, the support element is in the form of a cartridge 16 which is insertable into the slot 8 as shown by the arrows 18. The cartridge 16 has a base 20 and inwardly sloping edges 22, 24, which slope inwardly an angle corresponding to the angle of the inwardly sloping walls 12, 14. The cartridge 16 is configured such that when the cartridge is placed in the slot 8, the inwardly sloping edges 22, 24 register with the obliquely configured inwardly sloping sidewalls 12, 14 to anchor the cartridge 16 in position and prevent it from disengaging from the slot 8. The base 20 of the cartridge 16 may be provided with an adherent surface to further anchor the cartridge in the slot 8.

In FIG. 1b, it will be seen that the slot 8 is not as deep as the slot 8 in FIG. 1a. This difference in slot depth permits cartridges 16 of different material loading to be employed in the device, as shown in FIGS. 1e and 1f.

The cartridge 16 is provided with a material 26 be administered internally of the body cavity into which the elongate body is to be inserted. The material 26 is typically a medicament in the form of an ointment. In the embodiment shown in FIG. 1e, the medicament may be impregnated in a plastic or cloth material such as felt 27 that is stretched provided on an upper surface of the cartridge. In the embodiment shown in FIG. 1f, the medicament 26 is provided as a more viscous jelly on the surface of the cartridge and is can be removed by friction as it reaches the fissure.

In use of the device illustrated in FIG. 1, a suitable medication is provided on the support element and the support element is inserted in the support element receiving zone. The elongate body is oriented to position the aperture in regard to the site of application and applicator is inserted in the body cavity to apply the medication to the site of application.

The medicament typically employed in the treatment of a fissure is a nitroglycerin ointment. The ointment has a viscosity such that it will stick to the cartridge sufficiently that it does not become removed from the cartridge during entry into the cavity and prior to reaching the point of application at the site of the fissure.

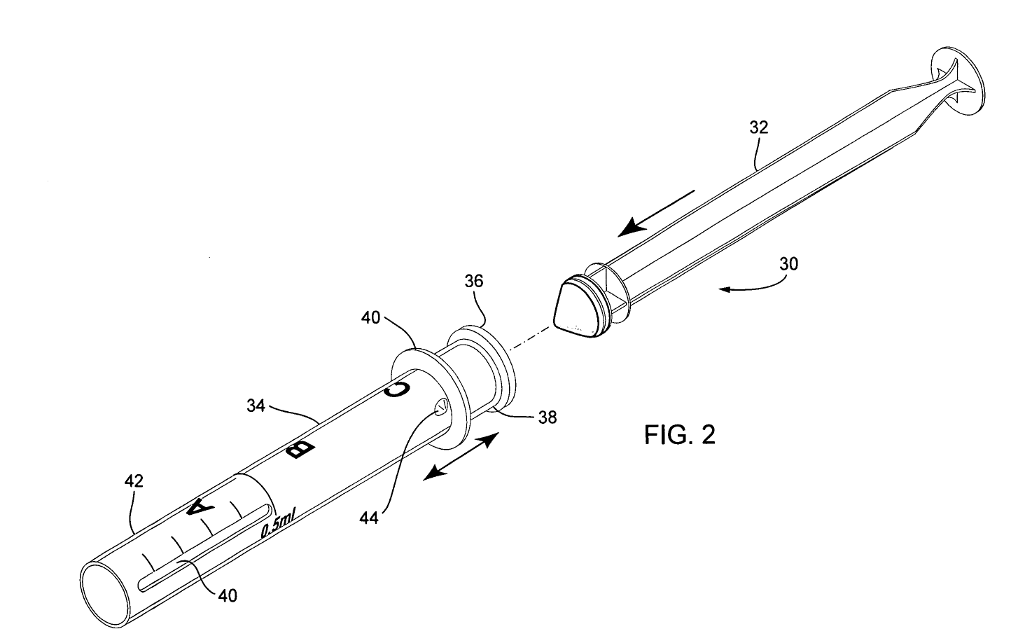

In FIG. 2, there is shown a syringe embodiment 30 having a moveable element 32 and an elongate body in the form of a cylinder 34 for receiving the moveable element 32, typically in the form of a plunger. The cylinder 34 is provided with a circumferential barrier 36 at the proximate end 38 and a longitudinally extending aperture 40 at the distal end 42 of the cylinder, through which medication is extruded onto the site of application within the body cavity upon application of manual pressure to the plunger 32. A ring 40 is provided which slides longitudinally along the cylinder 34 between the barrier 36 and a marker 44, to mark the distance of insertion of the cylinder into the cavity. The marker 44 is typically in the form of a knurl and is aligned with the aperture 40 (shown as an elongate slot) to indicate to the user the orientation of the aperture when inside a cavity. The distance of insertion into a cavity is also marked by letters “A”, “B” and “C”

The amount of medication may vary according to the desired treatment regimen. Thus, the entire contents of the chamber may be administered at once (say 0.5 ml). Alternatively, lower amounts (such as 0.25 ml) may be provided and individually administered over several days. Another alternative is to vary the concentration of the active ingredient in the ointment composition, say 2% wt or 1% wt. Another alternative is to have the cartridges numbered by day 1, 2 and so on so that weaker doses can be applied in the early days and stronger doses later as the fissure heals and systemic absorption decreases.

In use of the device illustrated in FIG. 2, a suitable measured dose and concentration of medication is provided in the elongate body, which is then inserted into the body cavity. The elongate body is oriented to position the aperture in regard to the site of application, and the moveable element is urged inwardly of the elongate body to force medication in the interior space out through the aperture onto the site of application.

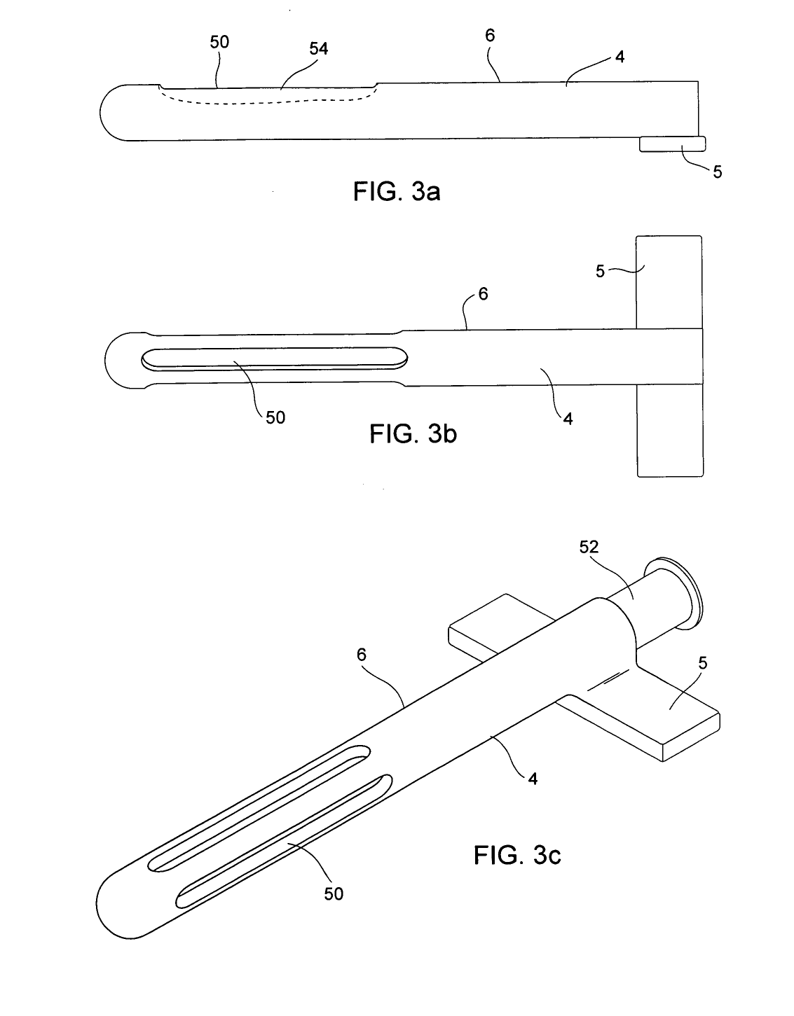

FIGS. 3a-3c show an alternative embodiment wherein longitudinally extending slots 50 are provided on the outer surface 6 of the elongate body 4 and a plunger 52. The slots 50 are typically equispaced around the external surface 6 of the elongate body 4. Medication 54 is located within the slots and is smeared onto the site of application upon insertion into the body cavity and manual depression of the plunger 52.

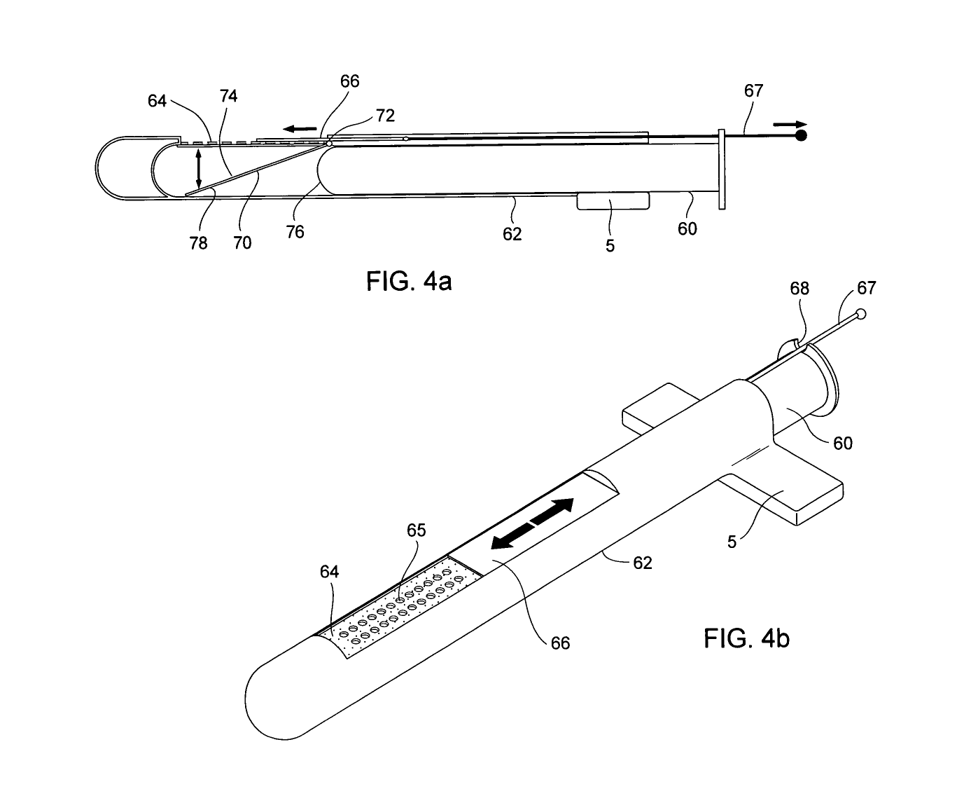

FIGS. 4a-4b show an alternative embodiment comprising a plunger 60 and a cylinder 62. The cylinder is provided with a removable apertured plate 64 containing a series of apertures 65 through which medication may be extruded upon manual depression of the plunger 60. The device is provided with a cover 66 which is moveable back and forth by way of handle 67 extending though notch 68 from a position which covers the apertures to one in which the apertures are open. Handle 5 and apertures 65 are positioned on the cylinder 62 such that, in use, when the handle 5 extends horizontally across the bottom of the cylinder, the apertures are disposed on the top of the cylinder. This allows the user to know the orientation of the apertures when not visible inside a cavity. The cylinder 62 is provided with an interior ramp 70 that is hinged at hinge point 72 and is adapted to receive medication on upper surface 74 thereof. Depression of the plunger 60 results in distal end 76 contacting the undersurface 78 of the ramp 70 and forcing the ramp upwardly about hinge point 72 to extrude medication on the upper surface 74 through the apertures 64 into contact with the site of application. Withdrawal of the plunger 60 allows the ramp 70 to return to its lower position as shown in FIG. 4a.

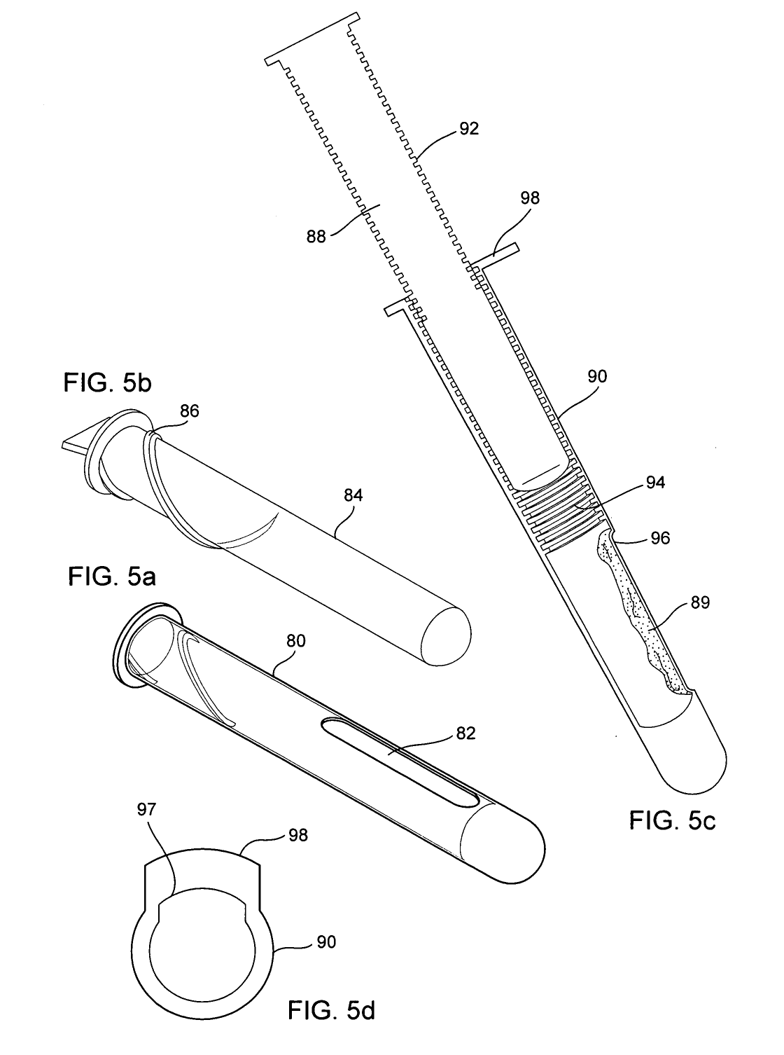

FIGS. 5a-5d show yet further alternative embodiments of the invention. FIG. 5a shows a cylinder 80 provided with a slot 82 through which medication is extruded upon application of pressure to the plunger 84 shown in FIG. 5b. The plunger 84 in FIG. 5b is provided with an external screw 86 that engages with a corresponding internal screw (not shown) located on an internal surface of the cylinder 80. The pitch of the screw will determine the rate of extrusion of medication through the slot 82 as the plunger is rotated. This permits metered dispensing of medication through the slot 82. FIG. 5c shows a further embodiment of a device comprising a plunger 88 and cylinder 90 with an aperture 96. The plunger 88 is provided with an external thread 92 that engages with a corresponding thread 94 provided on an internal surface of the cylinder 90. Rotation of the plunger 88 permits a metered amount of medication 89 to be extruded to the site of application through aperture 96. FIG. 5d shows the end view of the cylinder 90 with an enlarged flange 98 aligned with aperture 96 to indicate to the user the orientation of the aperture 96 when inside a cavity. A notch 97 may be provided which acts a rail to orient the plunger 88 in the cylinder if the shape of the tip requires this for optimal function.

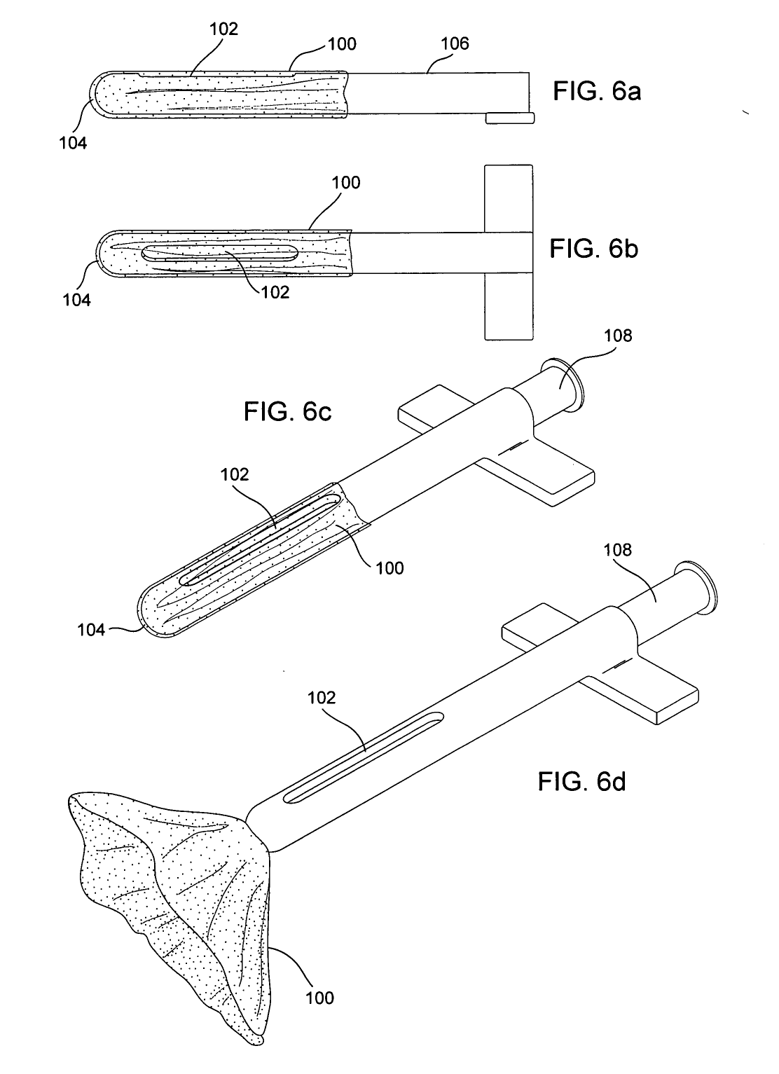

FIGS. 6a-6d show a further embodiment where a protective cover 100, for example saran wrap, is provided over elongate slots 102 and adhered at the distal end 104 of the cylinder 106. The protective cover 100 rolls back as shown in FIG. 6d as the cylinder 106 is withdrawn from the body cavity to expose the slots 102 and permit application of the medication to the affected site by depression of the plunger 108.

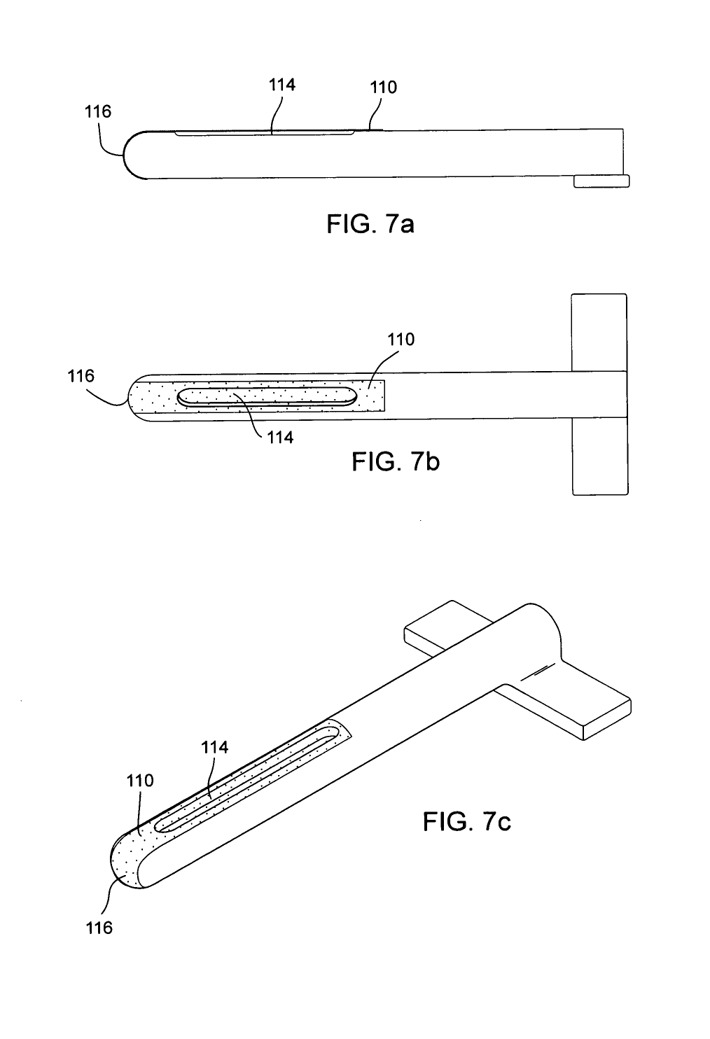

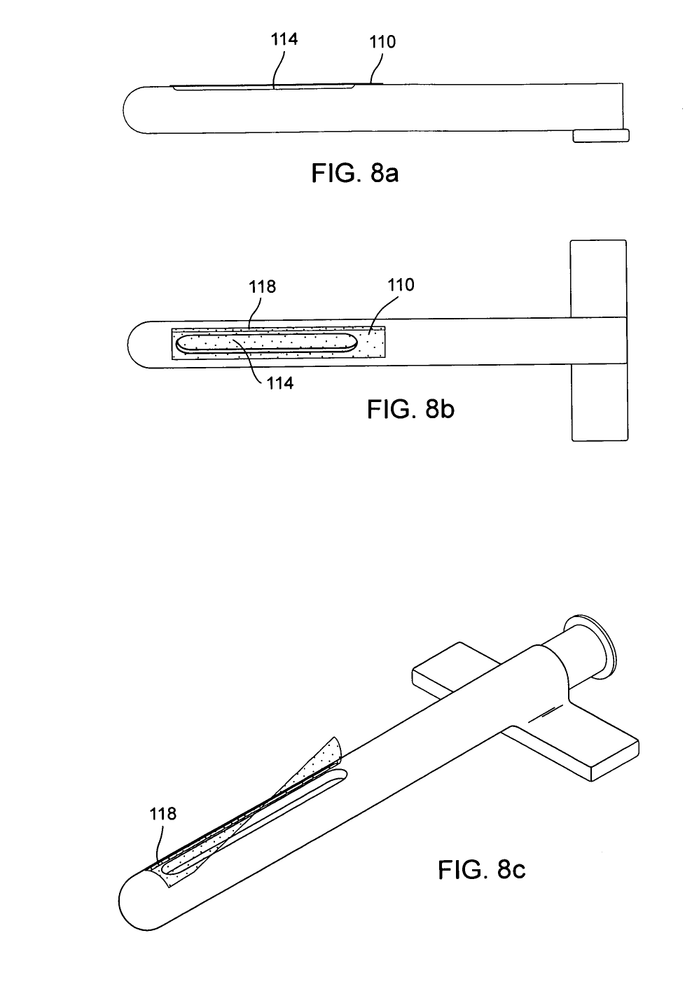

FIGS. 7a-7c and FIGS. 8a-8c show similar embodiments where a protective cover 110, 112 is provided over a slot 114. In FIGS. 7a-7c, the cover is adhered at the distal end 116, and rolls back from the slot when application of medication present in the slots to the affected site is desired.

In FIGS. 8a-8c, the cover is adhered along the edge 118. As shown in FIG. 8c, the cover peels away upon application of medication present in the slots to the affected site.

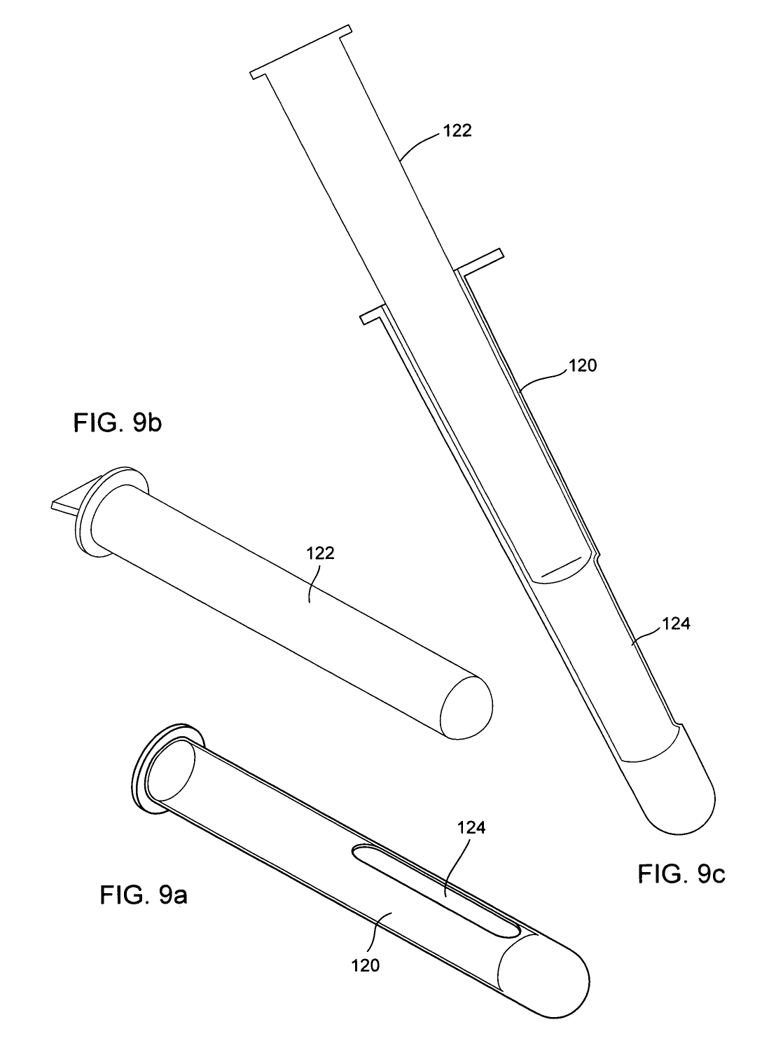

FIGS. 9a-9c show further embodiments comprising a cylinder 120 and plunger 122. The cylinder 120 is provided with a slot 124 through which medication is extruded from the interior of the cylinder upon depression of the plunger 122.

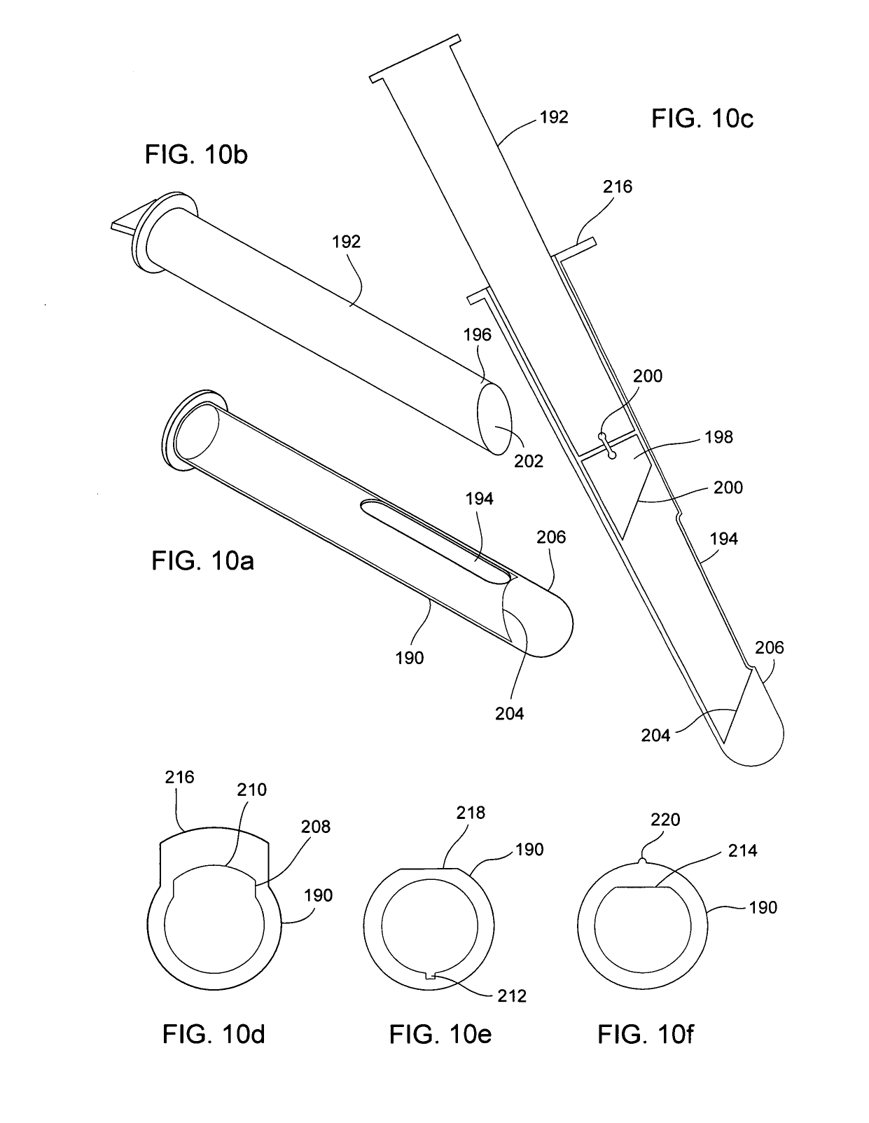

FIGS. 10a-10c show an embodiment comprising a cylinder 190 and plunger 192. The cylinder is provided with a slot 194 through which medication is extruded from the interior of the cylinder upon depression of the plunger 192. In this embodiment, the distal end 196 of the plunger 192 includes a separately rotatable wedge-shaped tip member 198 rotatably connected to the plunger by a linkage 200. The tip member 198 has a sloping wedge face 202 which comes into abutment with a corresponding internal sloping surface 204 at the distal end 206 of the cylinder 190 when the plunger 192 is urged inwardly of the cylinder.

The orientation of wedge-faced tip member 198 with respect to the cylinder is maintained by guide means provided on an inner surface of the cylinder 190. Examples of guide means are shown in FIGS. 10d-10f. In FIG. 10d, a track 208 is provided inside the cylinder with a concave arcuate surface 210 for accommodating a correspondingly shaped convex surface on the tip member 198. In FIG. 10e, a slot 212 is provided which registers with a corresponding projection provided on the tip member 198. In FIG. 10c, a flat portion 214 is provided in the inner surface of the cylinder 190 which registers with a corresponding flat portion provided on the tip member 198.

In order to be able to orient the cylinder 190 when the slot 194 is not visible (for example, when inside a body cavity), an indicator means is provided at the proximal end of the cylinder 190. For example, a flange 216 aligned with slot 194 may be provided (see FIG. 10d). Alternatively, the cylinder may be provided with a flat surface 218 or a protuberance 220, and shown in FIGS. 10e and 10f, respectively.

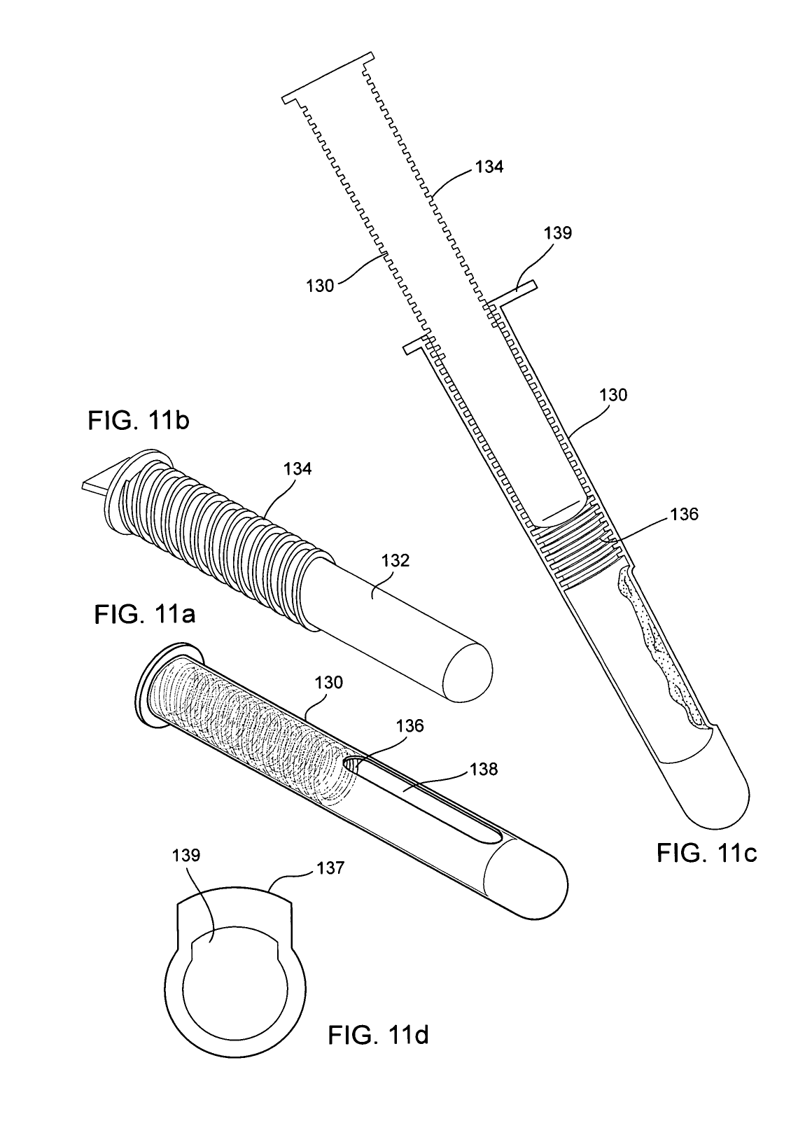

FIGS. 11a-11c show a further embodiment comprising a cylinder 130 and plunger 132 with a threaded portion 134 on an external surface of the plunger 132 that is engageable with a corresponding threaded portion 136 on the internal surface of the cylinder 130. If the plunger has a wedge shaped distal end, the plunger may have a configuration similar to that described in FIG. 10 where the tip is independently moveable with respect to the remainder of the plunger.

FIG. 11d shows a flange arrangement 137 that indicates to the user the orientation of the slot 138 through which ointment is dispensed when the slot is not visible (see FIG. 10d). As in FIG. 10d, an interior track 139 may be provided in the cylinder to maintain the plunger in a desired orientation within the cylinder so that the shaped tip is in optimal position for extruding the ointment through the slot 138. The screw feature permits accurate dispensation of metered amounts of medication to the affected site through slot 138 as the plunger is rotated into the cylinder.

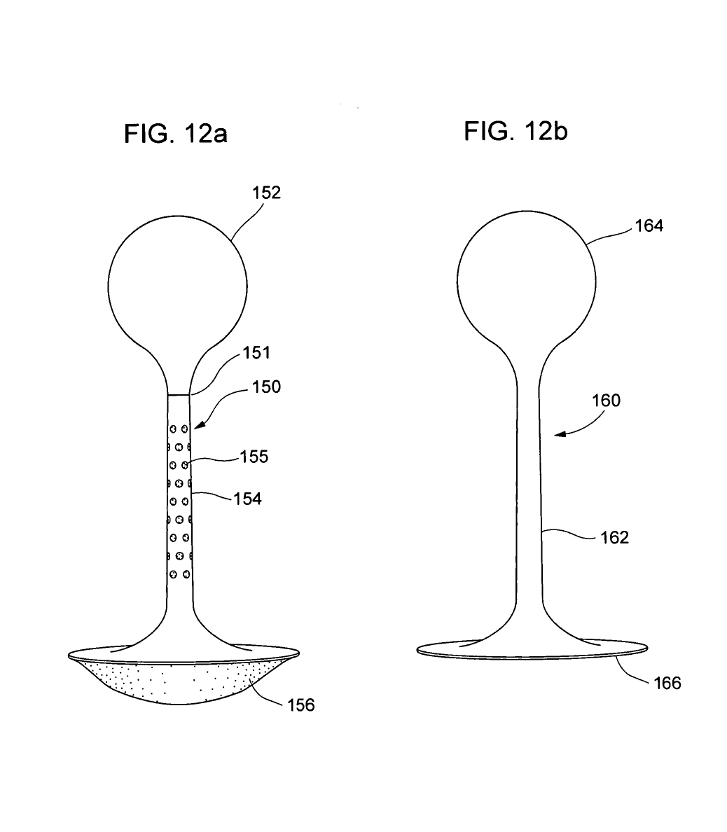

FIGS. 12a and 12b show suppository devices suitable for insertion into the anus. The device 150 of FIG. 12a has a spherical shaped distal end 152 and a stem or narrow portion 154 for containing a measured amount of medicament, such as nitroglycerine. The device 150 is provided at its proximal end with a pliable diaphragm 156. The stem 154 is partially hollow up to the region 151 for containing medication. The stem 154 is provided with apertures 155 extending circumferentially around the stem through which medication can be extruded from the interior of the stem. In use, the device 150 is inserted into the anus such that the upper rounded end 152 proceeds into the rectum, leaving the pliable diaphragm 156 protruding slightly from the anus, to maintain the device in position in the ampulla and prevent the device from migrating entirely into the rectum. By depressing the diaphragm 156, a measured amount of medicament is expressed directly into the anal canal in a circumferential manner through the apertures 155.

In FIG. 12b, the device 160 is similar in shape to that of FIG. 12a having a stem 162, a spherical distal end 164 and a flange-shaped proximal end portion 166. However, in this embodiment, the stem 162 is not hollow and is not provided with apertures. In use, the device 160 is inserted into the anus with the assistance of a plain lubricant applied to the bulb 164. A measured amount of the medicament (for example, nitroglycerin-containing ointment) is applied to the exterior of the stem 162 allowing the medicament to be deployed at the appropriate site to act on the fissure for a few minutes. When nitroglycerin ointment is employed, this dissolves over time and the active ingredient goes to the fissure in the anal canal. The shape of the device 160 is such that, as with the device 150, the bulb 164 is maintained in position in the ampulla, with the stem in the anal canal, and the end portion 166 prevents the device from entering further into the rectum and facilitates convenient withdrawal after administration of the medicament.

In use of the device of FIG. 12a, a suitable medication is provided in the stem followed by insertion of the applicator into the body cavity. Extrusion of medication through the apertures is achieved by depressing the diaphragm, to cause medication to be applied to the site of application. The device of FIG. 12b is used by applying medication onto the stem and then inserting the applicator into the body cavity, whereby medication present on the stem is delivered to the site of application.

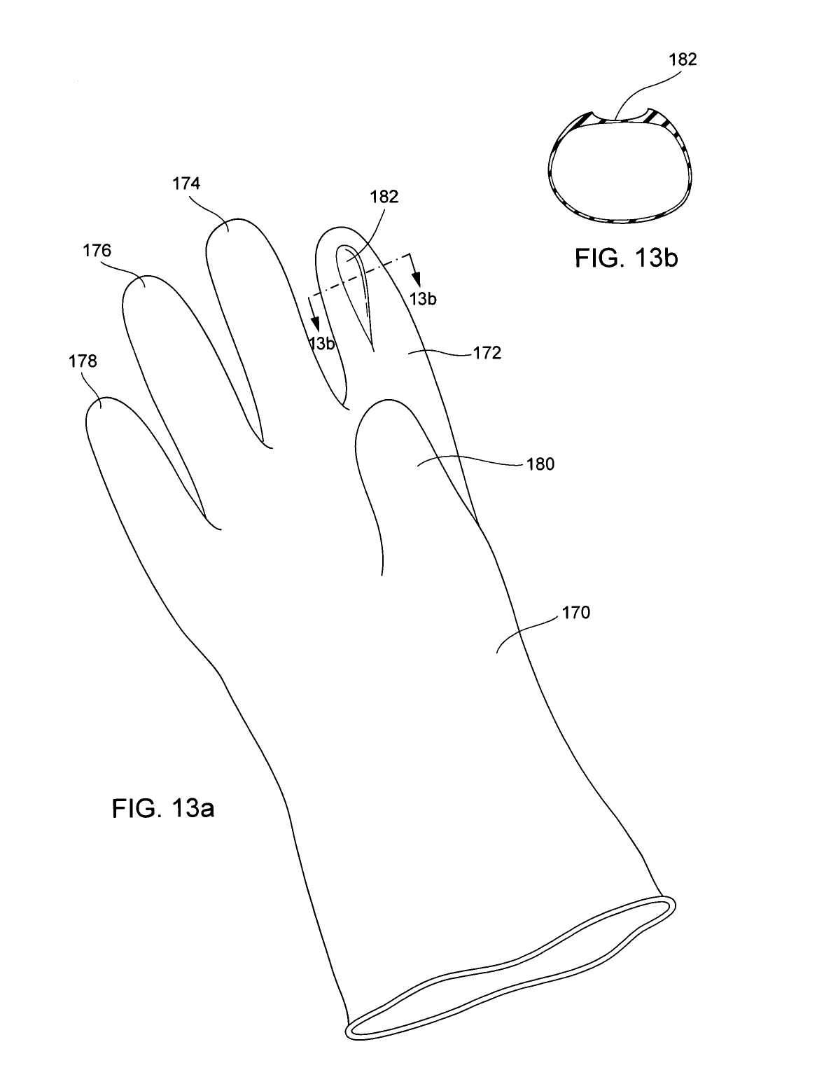

FIG. 13a shows a glove applicator 170 with finger elements 172, 174, 176, 178, 180 for receiving respective fingers and thumb of a user's hand. The forefinger element 172 is provided with a hollow 182 for receiving a measured amount-of ointment, for example about 0.5 ml. FIG. 13b is a cross-section of the forefinger element 172 along line A-A, and shows the hollow 182. In use, the glove applicator is placed on the user's hand and medication is introduced into the hollow 182. The forefinger element 172 is then inserted into the anus and the medication applied manually by smearing onto the fissure.

The anal canal is typically about 3 cm long and is surrounded by smooth muscle and usually held closed (the neck of the gourd). Fissures are typically linear in the long axis of the canal. About 95% are directly posterior, 3% are anterior, and the rest are both anterior and posterior or at another angle. The physician or informed patient easily determines the location.

The fissure may typically be up to 3 cm long. The devices of the present invention therefore are able to dispense the medication typically ointment to the fissure directly, and do not dispense ointment outside the anal canal. A simple syringe would not be effective in this situation because the syringe could easily dispense the ointment to the rectal ampulla and the patient would experience adverse side effects without the benefit of direct application of the medication to the fissure.

Additionally, the devices of the invention are able to deliver a certain measured dose of medication to the fissure. The dose can be varied in all of the devices described, either by altering the quantity and/or the concentration of medication delivered. An important consideration is that the dose of ointment can be increased or decreased depending on the rate of healing. The common situation is a lower dose to begin with and a higher dose as the healing progresses and the systemic absorption is decreased. Any of the devices described above can be reloaded, or a set of preloaded closed systems with variable doses and strengths for use over a period of time, for example a month.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1-33. (canceled)

34. A method of applying a medication to a site of application inside a body cavity, said method comprising the steps of:
(a) providing an applicator suitable for delivering a measured dose and concentration of medicinal material to a body cavity comprising an elongate support body having an external surface and a blunt distal end to facilitate insertion into a body cavity and a support element receiving zone located towards said distal end for receiving said support element, said support element receiving zone being configured to receive a correspondingly shaped support element for carrying medicinal material, whereby upon insertion of said elongate body into said body cavity, medicinal material supported on a support element located in said receiving zone is released to a desired location within said cavity;
(b) providing a suitable medication on said support element;
(c) inserting said support element in said support element receiving zone;
(d) orienting said elongate body to position said aperture in regard to said site of application;
(e) inserting said applicator in said body cavity to apply said medication to said site of application.

35. A method of applying a medication to a site of application inside a body cavity, said method comprising the steps of:
(a) providing an applicator suitable for delivering medicinal material to a body cavity comprising a hollow elongate body having a wall defining an interior space, said wall having an external surface and a blunt distal end to facilitate insertion into a body cavity, an aperture extending through said wall, said aperture being located near said distal end, a marker aligned with said aperture to indicate to a user the orientation of the aperture when not visible to the user, and a moveable element disposed within said interior space for forcing material in said interior space out through said aperture to a site of application;
(b) providing a suitable measured dose and concentration of medication in said elongate body;
(c) inserting said applicator into said body cavity;
(d) orienting said elongate body to position said aperture in regard to said site of application;
(e) urging said moveable element into said elongate body to force material in said interior space out through said aperture to said site of application.

36. A method of applying a medication to a site of application inside a body cavity, said method comprising the steps of:
(a) providing an applicator suitable for delivering a measured dose and concentration of medicinal material to a body cavity comprising a bulbous distal end, a flexible diaphragm at its proximal end and a hollow stem having at least one aperture extending between said bulbous end and said diaphragm;
(b) providing a suitable medication in said stem;
(c) inserting said applicator into said body cavity;
(d) depressing said diaphragm to extrude medication through said at least one aperture to said site of application.

37. A method of applying a medication to a site of application inside a body cavity, said method comprising the steps of:
(a) providing an applicator suitable for delivering a measured dose and concentration of medicinal material to a body cavity comprising a bulbous distal end, a flange at its proximal end and a stem extending between said bulbous end and said flange;
(b) providing a suitable medication on said stem;
(c) inserting said applicator into said body cavity, whereby medication present on said stem is delivered to a site of application.

38. A method of applying a measured dose and concentration of medication to a site of application inside a body cavity, said method comprising the steps of:
(a) providing a glove applicator for delivering medicinal material to a body cavity comprising finger elements for receiving at least one finger and a thumb of a user's hand, one of said finger elements having a hollow for receiving a measured amount of medication;
(b) providing a suitable measured dose and concentration of medication in said hollow;
(c) inserting said finger element with said hollow containing medication into said body cavity, whereby the medication is applied to the site of application.
